(19) United States
(12) Patent Application Publication
(10) Pub. No.: US 2006/0229210 A1
(43) Pub. Date: Oct. 12, 2006

(54) Carrier based on granules produced from pyrogenically prepared silicon dioxides

(76) Inventors: Peter Neugebauer, Offenbach (DE); Steffen Hasenzahl, Hanau (DE); Claus-Peter Drexel, Neuisenburg (DE); Jurgen Meyer, Stockstadt (DE)

Correspondence Address: SMITH, GAMBRELL & RUSSELL, SUITE 3100, PROMENADE II, 1230 PEACHTREE STREET, N.E., ATLANTA, GA, 30307-3592, US

(21) Appl. No.: 10/568,012

(22) PCT Filed: Jun. 22, 2004
(86) PCT No.: PCT/EP04/06719

§ 371 Date: Feb. 10, 2006

(30) Foreign Application Priority Data
Aug. 13, 2003 (DE) 10337198.2

(51) Int. Cl. A01N 25/12; A61K 8/02; A01N 25/00; C04B 14/04

(52) U.S. Cl. 504/367; 424/405; 106/482; 424/401

(57) ABSTRACT

Carrier based on granules produced from pyrogenically prepared silicon dioxides. The use of granules produced from pyrogenically prepared silicon dioxides as carriers for substances including foodstuffs additives, such as dyestuffs, antioxidants, preservatives, emulsifiers, gelling agents, thickeners and binders, stabilizers, alkalis, acids, salts, antilumping agents, flavour intensifiers, sweeteners, aromas; feedstuff additives; chemical intermediates and plant protection agents.

The present invention relates to the use of granules of pyrogenic silica as carriers. In addition to various other actions, the granules can have the function of a carrier for foodstuffs additives, such as dyestuffs, antioxidants, preservatives, emulsifiers, gelling agents, thickeners and binders, stabilizers, alkalis, acids, salts, antilumping agents, flavour intensifiers, sweeteners, aromas, feedstuffs additives, chemical intermediates and plant protection agents, such as, for example, herbicides, insecticides, fungicides and others.

It is known to employ spherical silicon dioxide particles as carriers, for example for feedstuffs additives (Sipernat 22, Bulletin Pigments No. 31, “Synthetic Silica as a Flow Acid and Carrier Substance”, Degussa AG).

Disadvantages of the abovementioned silicon dioxide particles which are employed as carriers are their high water content, their too low purity and the poor flow properties of the loaded substance. Silicic acid esters, silica sols or silicates are employed as starting compounds, and then often lead to products of which the purity is not adequate for the desired intended uses because of considerable amounts of salts, so that an expensive washing is necessary.

The invention is therefore based on the object of providing spherical silicon dioxide particles for use as carriers which do not have the disadvantages mentioned and moreover meet the high demands of uses in respect of purity, product safety and flow properties.

The invention provides the use of granules based on pyrogenically prepared silicon dioxide as a carrier for substances chosen from the group consisting of foodstuffs additives, such as dyestuffs, antioxidants, preservatives, emulsifiers, gelling agents, thickeners and binders, stabilizers, alkalis, acids, salts, antilumping agents, flavour intensifiers, sweeteners, aromas, feedstuffs additives, chemical intermediates and plant protection agents, such as herbicides, insecticides, fungicides and others.

The invention also provides an adsorbate of granules based on pyrogenically prepared silicon dioxide and at least one substance chosen from the group consisting of foodstuffs additives, such as dyestuffs, antioxidants, preservatives, emulsifiers, gelling agents, thickeners and binders, stabilizers, alkalis, acids, salts, antilumping agents, flavour intensifiers, sweeteners, aromas, feedstuffs additives, chemical intermediates and plant protection agents, such as herbicides, insecticides and fungicides.

The granules based on pyrogenically prepared silicon dioxide preferably have an average particle diameter of 10 to 120 μm and a BET surface area of 40 to 400 m²/g (determination in accordance with DIN 66 131 with nitrogen).

The silicon dioxide granules furthermore preferably have the following physico-chemical characteristic data, which are determined as described in EP PS 0 725 037:

Pore volume: 0.5 to 2.5 ml/g

Pore size distribution: less than 5% of the total pore volume has a pore diameter of less than 5 nm, remainder meso- and macropores

pH: 3.6 to 8.5

Tamped density: 220 to 700 g/l.

Granules which are suitable for the use according to the invention and the preparation thereof are described, for example, in EP OS 0 727 037.

The granules can preferably have meso- and macropores, the volume of the mesopores making up 10 to 80% of the total volume. The particle size distribution of the granules is preferably 80 vol. % larger than 8 μm and 80 vol. % smaller than 96 μm. In a preferred embodiment of the invention, the content of pores smaller than 5 μm is not more than 5%, based on the total pore volume.

The granules employed according to the invention can be prepared, for example, by dispersing pyrogenically prepared silicon dioxide, preferably silicon dioxide prepared from silicon tetrachloride by means of flame hydrolysis, in water, spray drying the dispersion and optionally then heat-treating the resulting granules at a temperature of 150 to 1,100° C. for a period of 1 to 8 h.

The dispersion in water preferably has a concentration of silicon dioxide of 5 to 25 wt. %, more preferably 5 to about 19.9 wt. %. The spray drying can be carried out at a temperature of 200 to 600° C., and disc atomizers or nozzle atomizers can be employed in this context. The heat treatment of the granules can be carried out either in a static bed, such as, for example, in chamber ovens, or in an agitated bed, such as, for example, rotary tubular dryers.

The pyrogenic silicon dioxide serving as the starting compound is prepared by a process in which a volatile silicon compound is injected into an oxyhydrogen gas flame of hydrogen and air. Silicon tetrachloride is used in most cases. This substance hydrolyses to silicon dioxide and hydrochloric acid under the influence of the water formed during the oxyhydrogen gas reaction. After leaving the flame the silicon dioxide enters into a so-called coagulation zone, in which the silicon dioxide primary particles and primary aggregates agglomerate. The product present as a type of aerosol in this stage is separated from the gaseous concomitant substances in cyclones and then after-treated with damp hot air. The residual hydrochloric acid content can be lowered to below 0.025% by this process.

The granules based on pyrogenically prepared silicon dioxide can be silanized. The carbon content of the granules is then preferably 0.3 to 15.0 wt. %. Halogenosilanes, alkoxysilanes, silazanes and/or siloxanes can be employed for the silanization.

The following substances can be employed in particular as halogenosilanes:

Halogeno-organosilanes of the type X₃Si(CₙH₂ₙ₊₁)

X=Cl, Br
n=1-20
   Halogeno-organosilanes of the type X₂(R′)Si(CₙH₂ₙ₊₁)
X=Cl, Br
R′=alkyl
n=1-20
   Halogeno-organosilanes of the type X(R′)₂Si(CₙH₂ₙ₊₁)
X=Cl, Br
R′=alkyl
n=1-20
   Halogeno-organosilanes of the type X₃Si(CH₂)ₘ—R′
x=Cl, Br
m=0.1-20
R′=alkyl, aryl (e.g. —C₆H₅)
—C₄F₉, —OCF₂—CHF—CF₃, —C₆F₁₃, —O—CF₂—CHF₂
—NH₂, —N₃, —SCN, —CH═CH₂,
—OOC(CH₃)C═CH₂
—OCH₂—CH(O)CH₂ 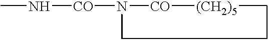
—NH—COO—CH₃, —NH—COO—CH₂—CH₃, —NH—(CH₂)₃Si(OR)₃
—Sₓ—(CH₂)₃Si(OR)₃
   Halogeno-organosilanes of the type (R)X₂Si(CH₂)ₘ—R′
X=Cl, Br
R=alkyl
m=0.1-20
R′ alkyl, aryl (e.g. —C₆H₅)
—C₄F₉, —OCF₂—CHF—CF₃, —C₆F₁₃, —O—CF₂—CHF₂
—NH₂, —N₃, —SCN, —CH═CH₂,
—OOC(CH₃)C═CH₂
—OCH₂—CH(O)CH₂ 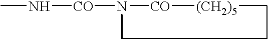
—NH—COO—CH₃, —NH—COO—CH₂—CH₃, —NH—(CH₂)₃Si(OR)₃
—Sₓ—(CH₂)₃Si(OR)₃
   Halogeno-organosilanes of the type (R)₂XSi(CH₂)ₘ—R′
X=Cl, Br
R=alkyl
m=0.1-20
R′=alkyl, aryl (e.g. —C₆H₅)
—C₄F₉, —OCF₂—CHF—CF₃, —C₆F₁₃, —O—CF₂—CHF₂
—NH₂, —N₃, —SCN, —CH═CH₂,
—OOC(CH₃)C═CH₂
—OCH₂—CH(O)CH₂ 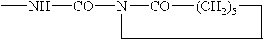
—NH—COO—CH₃, —NH—COO—CH₂—CH₃, —NH—(CH₂)₃Si(OR)₃
—Sₓ—(CH₂)₃Si(OR)₃

The following substances can be employed in particular as alkoxysilanes:
Organosilanes of the type (RO)₃Si(CₙH₂ₙ₊₁)
R=alkyl
n=1-20
   Organosilanes of the type R′ₓ(RO)_{y}Si(CnH₂ₙ₊₁)
R=alkyl
R′=alkyl
n=1-20
x+y=3
x=1.2
y=1.2
   Organosilanes of the type (RO)₃Si(CH₂)ₘ—R′
R=alkyl
m=0.1-20
R′=alkyl, aryl (e.g. —C₆H₅)
—C₄F₉, OCF₂—CHF—CF₃, —C₆F₁₃, —O—CF₂—CHF₂
—NH₂, —N₃, —SCN, —CH═CH₂,
—OOC(CH₃)C═CH₂
—OCH₂—CH(O)CH₂ 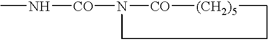
—NH—COO—CH₃, —NH—COO—CH₂—CH₃, —NH—(CH₂)₃Si(OR)₃
—Sₓ—(CH₂)₃Si(OR)₃
   Organosilanes of the type (R″)ₓ(RO)_{y}Si(CH₂)ₘ—R′
R=alkyl
x+y=2
x=1.2
y=1.2
R′=alkyl, aryl (e.g. —C₆H₅)
—C₄F₉, —OCF₂—CHF—CF₃, —C₆F₁₃, —O—CF₂—CHF₂
—NH₂, —N₃, —SCN, —CH═CH₂,
—OOC(CH₃)C═CH₂
—OCH₂—CH(O)CH₂ 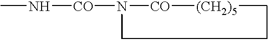
—NH—COO—CH₃, —NH—COO—CH₂—CH₃, —NH—(CH₂)₃Si(OR)₃
—Sₓ—(CH₂)₃Si(OR)₃

The silane Si 108 [(CH₃O)₃—Si—C₈H₁₇]trimethoxyoctylsilane can preferably be employed as the silanizing agent.

The following substances can be employed in particular as silazanes:
Silazanes of the type: 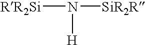
R=alkyl
R′=alkyl, vinyl
   and, for example, hexamethyldisilazane.

The following substances can be employed in particular as siloxanes:
Cyclic polysiloxanes of the type D 3, D 4, D 5, e.g. octamethylcyclotetrasiloxane=D 4 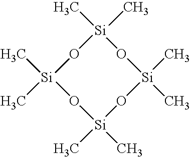
Polysiloxanes or silicone oils of the type: 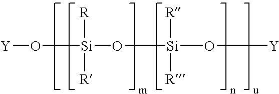
R=alkyl, aryl, (CH₂)ₙ—NH₂, H
R′=alkyl, aryl, (CH₂)ₙ—NH₂, H
R″=alkyl, aryl, (CH₂)ₙ—NH₂, H
R″′=alkyl, aryl, (CH₂)ₙ—NH₂, H
Y═CH₃, H, CₙH₂ₙ₊₁ where n=1-20
Y=Si(CH₃)₃, Si(CH₃)₂H
Si(CH₃)₂OH, Si(CH₃)₂(OCH₃)
Si(CH₃)₂(CₙH₂ₙ₊₁) where n=1-20
m=0, 1, 2, 3, . . . ∞
n=0, 1, 2, 3, . . . ∞
u=0, 1, 2, 3, . . . ∞

The silanization can be carried out by a procedure in which the granules are sprayed with the silanizing agent, which can optionally be dissolved in an organic solvent, such as, for example, ethanol, and the mixture is then heat-treated at a temperature of 105 to 400° C. over a period of 1 to 6 h.

An alternative method of the silanization of the granules can be carried out by a procedure in which the granules are treated with the silanizing agent in vapour form and the mixture is then heat-treated at a temperature of 200 to 800° C. over a period of 0.5 to 6 h. The heat treatment can be carried out under an inert gas, such as, for example, nitrogen.

The silanization can be carried out continuously or batchwise in heatable mixers and dryers with spray devices. Suitable devices can be, for example: plough share mixers or plate, fluidized bed or flow-bed dryers.

By varying the starting substances, the conditions during spraying, the heat treatment and the silanization, the physico-chemical parameters of the granules, such as the specific surface area, the particle size distribution, the pore volume, the tamped density and the silanol group concentration, pore distribution and pH, can be modified within the stated limits.

The invention also provides:
Dyestuff comprising granules based on pyrogenically prepared silicon dioxide.
Antioxidant comprising granules based on pyrogenically prepared silicon dioxide.
Preservative comprising granules based on pyrogenically prepared silicon dioxide.
Emulsifier comprising granules based on pyrogenically prepared silicon dioxide.
Gelling agent comprising granules based on pyrogenically prepared silicon dioxide.
Thickener comprising granules based on pyrogenically prepared silicon dioxide.
Binder comprising granules based on pyrogenically prepared silicon dioxide.
Stabilizer comprising granules based on pyrogenically prepared silicon dioxide.
Alkali comprising granules based on pyrogenically prepared silicon dioxide.
Acids comprising granules based on pyrogenically prepared silicon dioxide.
Salts comprising granules based on pyrogenically prepared silicon dioxide.
Antilumping agent comprising granules based on pyrogenically prepared silicon dioxide.
Flavour intensifier comprising granules based on pyrogenically prepared silicon dioxide.
Sweetener comprising granules based on pyrogenically prepared silicon dioxide.
Aroma comprising granules based on pyrogenically prepared silicon dioxide.
Feedstuffs additives comprising granules based on pyrogenically prepared silicon dioxide.
Chemical intermediates comprising granules based on pyrogenically prepared silicon dioxide.
Plant protection agents comprising granules based on pyrogenically prepared silicon dioxide.
Herbicides comprising granules based on pyrogenically prepared silicon dioxide.
Insecticides comprising granules based on pyrogenically prepared silicon dioxide.
Fungicides comprising granules based on pyrogenically prepared silicon dioxide.

Foodstuffs additives can be:

Dyestuffs, such as, for example:
E100 Curcumin
E101 Riboflavin, Lactoflavin
E102 Tartrazine
E104 Quinoline Yellow
E110 Sunset Yellow S (azo dyestuff)
E120 Carminic acid, Cochineal
E122 Azorubine (azo dyestuff)
E123 Amaranth (azo dyestuff)
E124 Ponceau 4R (azo dyestuff)
E127 Erythrosine
E131 Patent Blue V
E132 Indigotine, Indigo Carmine
E140 Chlorophylls a+b
E141 Chlorophylls und Chlorophyllins, copper complexes
E142 Acid Brilliant Green BS
E150 Caramel, Sugar colour, Rum colour
E151 Brilliant Black BN (azo dyestuff)
E153 Charcoal, medicinal
E160 Carotenoids
E160a Beta-Carotene, Gamma-Carotene
E160b Bixin, Norbixin, (Annatto), Orlean
E160c Capsanthin, Capsorubin
E160d Lycopene
E160e Beta-Apo-8′-Carotenal (C30)
E160f Beta-Apo-8′-Carotenoic Acid Ethyl Ester
E161 Xanthophylls
E161a Flavoxanthin
E161b Lutein
E161c Cryptoxanthin
E161d Rubixanthin
E161e Violaxanthin
E161f Rhodoxanthin
E161g Canthaxanthin
E162 Betanin, Beetroot Red
E163 Anthocyan
E172 Iron oxide, iron hydroxide
E173 Aluminium
E174 Silver
E175 Gold
E180 Pigment Rubine BK, Lithol Rubine

Antioxidants can be:
E220 Sulfurous acid, sulfur dioxide
E221 Sodium sulfite
E222 Sodium hydrogen sulfite
E223 Sodium disulfite
E224 Potassium disulfite
E300 Ascorbic acid
E301 Sodium ascorbate
E302 Calcium ascorbate
E304 Ascorbyl palmitate
E306 Tocopherol-containing extracts of natural origin
E307 alpha-Tocopherol
E308 gamma-Tocopherol
E309 delta-Tocopherol
E310 Propyl gallate
E311 Octyl gallate
E312 Dodecyl gallate
E320 Butylhydroxyanisole (BHA)
E321 Butylhydroxytoluene (BHT)
E330 Citric acid
E331 Sodium citrate
E332 Potassium citrate
E333 Calcium citrate
E472c Citric acid esters Ethoxiquin

Preservatives can be:
E200 Sorbic acid
E201 Sodium sorbate
E202 Potassium sorbate
E203 Calcium sorbate
E210 Benzoic acid
E211 Sodium benzoate
E212 Potassium benzoate
E213 Calcium benzoate
E214 Ethyl 4-hydroxybenzoate
E215 Ethyl 4-hydroxybenzoate, sodium salt
E216 Propyl 4-hydroxybenzoate
E217 Propyl 4-hydroxybenzoate, sodium salt
E218 Methyl 4-hydroxybenzoate
E219 Methyl 4-hydroxybenzoate, sodium salt
E220 Sulfurous acid, sulfur dioxide
E221 Sodium sulfite
E222 Sodium hydrogen sulfite
E223 Sodium disulfite
E224 Potassium disulfite
E236 Formic acid
E280 Propionic acid
E281 Sodium propionate
E282 Calcium propionate
E283 Potassium propionate

Emulsifiers can be:
E322 Lecithin
E442 Ammonium salts of phosphatidic acids
E471 Edible fatty acids, mono- and diglycerides
E472 Esters of E471
E472a Acetic acid esters
E472b Lactic acid esters
E472c Citric acid esters
E472d Tartaric acid esters
E472e Diacetyltartaric acid esters
E472f Tartaric-acetic acid esters
E473 Sucrose esters of edible fatty acids
E474 Sugar glycerides
E475 Polyglycerol esters of edible fatty acids
E476 Polyglycerol esters of polycondensed ricinoleic acid
E477 Propylene glycol esters of edible fatty acids
E481 Sodium stearoyllactylate
E482 Calcium stearoyllactylate
E487 Sdoium lauryl sulfate

Gelling agents, thickeners and binders and stabilizers can be:
E400 Alginic acid
E401 Sodium alginate
E402 Potassium alginate
E403 Ammonium alginate
E404 Calcium alginate
E405 Propylene glycol alginate
E406 Agar-agar
E407 Carrageenan
E410 Carob bean flour
E412 Guar flour
E413 Tragacanth
E414 Gum arabic
E415 Xanthan
E416 Karaya gum
E417 Tara gum
E440 Pectins
E460a Cellulose, microcrystalline
E460b Cellulose, powdered
E461 Methylcellulose
E463 Hydroxypropylcellulose
E464 Hydroxypropylmethycellulose
E465 Methylethylcellulose
E466 Carboxymethylcellulose
E551 Silicon dioxide
E1411 Di-starch phosphate I
E1412 Di-starch phosphate II
E1413 Di-starch phosphate, phosphated
E1414 Di-starch phosphate, acetylated
E1420 Mono-starch acetate I
E1421 Mono-starch acetate II
E1422 Di-starch adipate, acetylated
E1423 Di-starch glycerol, acetylated
E1430 Di-starch glycerol
E1440 Hydroxypropyl-starch
E1441 Hydroxypropyl-di-starch glycerol
E1442 Hydroxypropyl-di-starch phosphate.

Alkalis, acids and salts can be:
E170 Calcium carbonate
E260 Acetic-acid
E261 Potassium acetate
E262 Sodium diacetate
E263 Calcium diacetate
E270 Lactic acid
E296 Malic acid
E325 Sodium lactate
E326 Potassium lactate
E327 Calcium lactate
E330 Citric acid
E331 Sodium citrate
E332 Potassium citrate
E333 Calcium citrate
E334 Tartaric acid
E335 Sodium tartrate
E336 Potassium tartrate
E337 Potassium sodium tartrate
E354 Calcium tartrate
E338 Orthophosphoric acid
E339 Sodium orthophosphate
E340 Potassium orthophosphate
E341 Calcium orthophosphate
E343 Magnesium orthophosphate
E350 Sodium malate
E351 Potassium malate
E352 Calcium malate
E450 Salts of di-, tri- and polyphosphoric acid (di-, tri- and polyphosphates)
E500 Sodium carbonate
E501 Potassium carbonate
E503 Ammonium carbonate
E504 Magnesium carbonate
E507 Hydrochloric acid
E508 Potassium chloride
E509 Calcium chloride
E510 Ammonium chloride
E514 Sodium sulfate
E515 Potassium sulfate
E516 Calcium sulfate
E524 Sodium hydroxide
E525 Potassium hydroxide
E526 Calcium hydroxide
E527 Ammonium hydroxide
E528 Magnesium hydroxide
E529 Calcium oxide
E530 Magnesium oxide
E541 Sodium aluminium phosphate
E574 Gluconic acid
E575 Glucono-delta-lactone
E576 Sodium gluconate
E577 Potassium gluconate
E578 Calcium gluconate

Antilumping agents can be:
E170 Calcium carbonate
E341 Calcium orthophosphate
E470 Edible fatty acids, sodium, potassium and calcium salts
E504 Magnesium carbonate,
E535 Sodium ferrocyanide
E536 Potassium ferrocyanide
E538 Calcium ferrocyanide

Flavour intensifiers can be:
E621 Sodium glutamate
E622 Potassium glutamate
E623 Calcium glutamate
E624 Magnesium glutamate
E625 Ammonium glutamate
E627 Sodium guanylate
E628 Potassium guanylate
E629 Calcium guanylate
E630 5′-Inosinic acid
E631 Sodium inosinate
E632 Potassium inosinate
E633 Calcium inosinate

Sweeteners can be:
E950 Acesulfame-K
E951 Aspartame
E952 Cyclamate
E954 Saccharin
E957 Thaumatin

Aromas:
Abriceine
Acetanisole cryst.
Acetophenone pure
Agar wood D50092NS
Agrumen aldehyde 6947L
Agrumex HC
Agrumex LC
Agrumovert 10897 C/J
Aldehyde C 6 nat.
Aldehyde C11 MOA
Aldehyde C12 MA
Aldehyde C14 so-called
Aldehyde C16 so-called
Aldehyde C18 so-called/Abricolin
Alcohol C 6 nat.
Alcohol C 8
Alcohol C 9
Alcohol C10
Alcohol C12
Allinat/Allyl isothiocyanate
Allinat/Allyl isothiocyanate (stab.)
Allyl caproate
Allyl caproate kosher
Allyl cyclohexylpropionate
Allyl heptylate
Allyl phenoxyacetate
Amarocit®
Ambre 83LN DB10028
Ambrebois D50407
Ambrettia C
Ambrettolide
Ambrinol S
Ambroxid cryst.
Ananas Coeur D50214
Anethol NPU 21/22° C.
Anethol supra 21.5° C.
Anisaldehyde pure
Anisyl alcohol
Anisole
Anisyl acetate
Apple 74180C PM
Apriconia 28855P extra PM
Baldrian Identoil B
Basilicum Synthessence
Bay Identoil
Benzalacetone
Benzaldehyde
Benzaldehyde dd
Benzophenone cryst.
Benzyl acetate
Benzyl acetone
Benzyl alcohol dd
Benzyl alcohol FR
Benzyl benzoate H&R
Benzyl benzoate M
Benzyl cinnamate
Benzyl formate
Benzyl propionate
Benzyl salicylate
Bergamot Identoil colourless
Bergamot Synthessence Afric.
Blackberry D50260E
Bois de Cachemire D50008
Bois Doux 78008SP PM
Boronal
Butyric acid nat.
Butyl butyrate
Cacao et Chocolat D50546B
Cajeput Identoil
Calmus Synthessence asarone-free
Cananga Identoil
Capric acid nat.
Caproic acid nat.
Caramel acetate
Cardamom R Identoil
Cardamom Synthessence
Cassia Identoil
Cassia Identoil B dark
Cassis D50060B
Cedar Leaves Identoil
Chloracetophenone para
Chrysantheme
Cinnamyl acetate.
Citral FF
Citron R
Citrone Synthessence FF
Citronella Identoil
Citronell Identoil
Citronellyl tiglinate
Citronitrile
Citrowanil® B
Citrozone D50620B
Citrylal
Citrylal E
Clarifruit D5 0757
Clarion Base D50774
Coriander Identoil
Corps 98N DB10025
Corps Racine VS
Costus Synthessence
Coumarone
Cumin Synthessence
Cypress Identoil
Damascenone beta nat. 1% in EtOH
Datilat
Decalactone gamma nat.
Decalyl acetate beta
Diacetyl nat.
Dibenzosuberenone
Dibenzosuberone
Dibenzyl ether
Diethyl phthalate (DEP)
Dihydrocoumarin
Dimethyl anthranilate
Dimethyl benzyl carbinyl butyrate
Dimethyl sulfide nat.
Diphenyl oxide
Silver Fir Needle Identoil
Silver Fir Needle Identoil B
Oak Moss Resin D50342
Strawberry D50026C
Acetic acid nat.
Estragon Identoil
Ethoxyfuranone
Ethyl 2-methylbutyrate nat.
Ethyl 2-methylbutyrate
Ethyl acetate nat.
Ethyl acetoacetate
Ethyl benzoate
Ethyl butyrate
Ethyl butyrate nat.
Ethyl caproate kosher
Ethyl caproate nat.
Ethyl caprylate
Ethyl caprylate nat.
Ethyl cinnamate
Ethyl formate
Ethyl heptylate
Ethyl isovalerate
Ethyl phenylacetate
Ethyl propionate
Ethyl salicylat
Eucalyptol
Eucalyptus Oil Globulus BP
Eugenol
Eugenol methyl ether
Farenal
Fennel oil techn.
Feuilles de Tomate 79569PM
Spruce Green 8001S
Spruce Needle Identoil B sib.
Spruce Needle Identoil sib.
Filbertone G
Fir Balsam DM
Fleur de Cassis SBU PM
Floropal
Florophyll 10183
Fragolane
Framboson 10583F
Frutinat
Galbanum Synthessence
Galbanum Synthresin B
Geranium Identoil Afric.
Geranium Identoil Bourbon
Geranyl tiglinate
Globalide 100%
Globanone 50% DEP
Globanone 50% DPG
Globanone 50% IPM
Grapefruit D50075N
Identoil D61286G Green Honey Melon D50315
Guave 10875N
Helichrysum Synthessence
Herbaflorat
Hexyl acetate
Hexyl acetate nat.
Hexyl salicylate
Hydrocitronitrile
Indian Spice 10898
Indoflor H&R cryst.
Indole FF
Ginger oil spec. D40393S
Ionone pure 100%
Iris Synthresin H&R
Irolene P
Isoamyl acetate G
Isoamyl acetate nat.
Isoamyl butyrate
Isoamyl butyrate nat.
Isoamyl isobutyrate nat.
Isoamyl isovalerate
Isoananate

Isobornyl acetate
Isobutyric acid nat.
Isobutyl acetat nat.
Isobutylquinoline
Isobutylquinoline 54
Isoeugenol methyl ether
Isotabac naturelle LN DB10038
Jasmaprunat
Camomile Identoil blue
Camomile rom. Synthessence
Pine Needle Identoil
Pine Needle Identoil B
Pine Needle Identoil B P
Kiwi D50195PM
Cresol methyl ether para
Lactojasmon
Lavandin Identoil 30/32%
Lavandin Identoil type French 30/32%
Lavandin Provence D50817
Lavender Identoil type Mt. Blanc 40/42%
Lavender oil type Mt. Blanc 40/42%
Leguminal
Limonene d pure
Loganberry D50398N PM
Bay Leaf Oil D50286
Mace Oil extra
Macrolide®
Macrolide® supra
Madranol
Magnolan
Majantol
Mandaril
Manderine Synthessence
Mango D50436PM
Maracuja D50042E PM
Marjoliane N DB10018
Mayciane N DB10023
Melissa Identoil German so-called
Menthol D dist.
Menthol liquid
Menthol rac.
Menthol rac. PH
Menthol-1 dest.
Menthol-1 H&R compacted
Menthol oil
Menthone-1/Isomenthone-d
Menthyl acetate-1
Metaxa D50247C
Methyl ethylpyrazine-2,3
Methyl 2-methylbutyrate
Methylacetophenone para
Methylacetophenone para supra
Methyl anthranilate
Methyl benzoate H&R
Methyl benzoate techn. pure
Methyl betanaphthyl ketone cryst.
Methylbutyric acid-2 nat.
Methyl cinnamate
Methyl phenylacetate
Methyl salicylate
Methyl cinnamaldehyde alpha
Miel Blanc N DB10024
Musk Seed Synthessence
Mugetanol
Mugofleur D50444PM
Clary Sage Identoil
Clary Sage Identoil B
Carnation Flower Identoil
Clove Leaf Identoil dark
Clove Leaf Oil deg.
Neononyl acetate
Neroli Identoil
Nerolin Yara Yara cryst.
Neroli oil 4663
Olibanum Synthresin
Orange Identoil TSA
Orange oil spec. D40393P
Origanum Identoil
Oryclon extra
Oryclon special
Osmanthia 353
Ozonil
Palisandal
Palisandin
Palmarosa Synthessence
Pastinak Synthessence
Patchouli Synthessence N
Patchouli oil deg. DM
Pear D50313A PM
Peru Balsam Identoil
Peru balsam art. H&R
Petitgrain Bigarade Synthessence
Petitgrain Identoil R
Peach D40110PM
Plum D50424
Phenirate
Phenoxyethyl alcohol/arosol
Phenylacetaldehyde 100%
Phenylacetaldehyde dimethyl acetal
Phenylethyl acetate
Phenylethyl alcohol benzyl alcohol-free
Phenylethyl alcohol pure
Phenylethyl cinnamate cryst.
Phenylethyl isobutyrate
Phenylethyl phenylacetate
Phenylpropyl alcohol
Pimento Identoil
Pineapple acetate
Poivre Coeur H&R PM
Poivron N DB10029
Prenyl acetate
Prenyl salicylate
Profarnesal
Projasmon P
Propionic acid nat.
Propyl acetate nat.
Prunol N DB10027
Pyroprunat
Rain Forest D50339C PM
Resedafol
Rosaphen
Rose Booster D50221A
Rose F50048R PG
Rosemary Identoil
Rosemary Identoil Spanish
Rosewood Braz. Identoil
Sage Identoil Span.
Sage Identoil Span.
Sandalwood S.E.A. D50820
Sandel 80
Sandel extra
Sandel Forte
Sandel H&R
Sandel H&R ECO
Sandel H&R super
Sandel SP
Sandel type East Ind.
Sandalwood type East Ind.
Sandolene H&R
Spike Identoil
Styrax Identoil D50186
Styrenyl acetate
Sweet Amber D50807
Tobacco aroma H&R D50799
Teatree D50780A
Thyme Identoil
Thyme red Identoil
Thyme Synthabsolue
Thymol dist.
Thymol cryst. H&R
Thymol cryst. PH
Tonca Synthresin
Vanillin nat.
Verbena Identoil type French
Verdeflora D50375D
Verdural F
Vertocitral
Vertocitral C
Vertosine
Vetiver Identoil J
Juniper berry Identoil 10900
Juniper berry Synthessence
Willow fragrance 6103CB HG
Wintergreen oil
Ylang 10372 MT
Ylang Ylang Identoil Bourbon I
Ylang Ylang Identoil Bourbon II
Ylang Ylang Identoil Bourbon III
Cinnamaldehyde
Cinnamaldehyde nat.
Cinnamyl alcohol
Cinnamon leaf Identoil
Cinnamon bark Identoil

Feedstuffs additives can be:
Choline chloride solution
Vitamin E acetate
Formic acid
Acetic acid
Propionic acid
Phosphoric acid
Fat concentrates
Ethoxiguin
Molasses
Hop extract
Tagetese extract
Lecithin
Whey
Calcium formiate
Urea
Milk substitute
Trace elements
Vitamins

Chemical intermediates can be:
1,2-Propylene glycol
Acrylic acid
Adipic acid
Adipic anhydride
Formic acid
Formic anhydride
Benzoic acid
Succinic acid
Butanoic acid
Butanoic anhydride
Caproic acid
Dimer fatty acid
Dimer fatty acid anhydride
Dipentaerythritol
Erucic acid
Acetic acid
Acetic anhydride
Ethylene glycol
Fumaric acid
Glutaric acid
Glycerol
Isophthalic acid
Isophthalic anhydride
Lauric acid
Linolenic acid
Linoleic acid
Maleic acid
Maleic anhydride
Malonic acid
Myristic acid
Oleic acid
Oxalic acid
Palmitic acid
Pentaerythritol
Phthalic acid
Phthalic anhydride
Propionic acid
Stearic acid
Terephthalic acid
Terephthalic anhydride
Trimethylolpropane
Valeric acid.
Bisphenol A
Epichlorohydrin
o-Cresol
Phenol novolaks
Styrene
α-Methylstyrene
Vinyltoluene
Methyl methacrylate
Divinylbenzene
Diallyl phthalate
Diisocyanates
Toluene-diisocyanates
Cyclohexanone
Methylcyclohexanone
Acetone
Butanone
Acetophenone
Indene
Coumarone (benzofuran)
2-Methylindene
2-Methylcoumarone
Methylstyrene
Cyclopentadiene
Dicyclopentadiene
Heteropolysaccharides
Arabinose
Galactose
Glucoronic acid
Mannose
Rhamnose
Xylose
Resinol acids.
Resinols
Resinotannols
Resenes
Terpenes
Diterpenes.
Triterpenes
Sesquiterpenes
Resin esters
Resin soaps
Alcohols
Phenol derivatives
Hydroquinone derivatives
Quinoline derivatives

Naturally occurring resins:
Acaroid resin
Asa foetida
Benzoin resin
Amber
Bitumen
Canada balsam
China lacquer
Copaiva balsam
Dammar resin
Dragon's blood resin
Elemi
Galbanum
Gutti
Jalap resin
Japan lacquer
Kauri copal
Colophony
Copal
Labdanum
Manila copal
Mastix
Myrrh
Olibanum
Opoponax
Pernambuco balsam
Peru balsam
Sandarac
Shellac
Styrax
Tolu balsam
Terpentine

Synthetic resins:
Hydrocarbon resins
Urea resins
Alkyd resins
Epoxy resins
Melamine resins
Melamine-formaldehyde resins
Hexamethylolmelamine resins
Melamine-phenol resins
Melamine-urea resins
Phenolic resins
Polyester resins
Unsaturated polyester resins
Polyurethane resins
Ketone resins
Coumarone-indene resins
Isocyanate resins
Polyamide resins
Terpene-phenol resins
Epoxy resins
Rubber

Additives:
Wetting agents
Desiccants
Antifloating agents
Antiskinning agents
Hardening accelerators
Hardening retardants
Expanding agents
Sealants
Water softeners
Deoxygenating agents
Buffers
Polishing agents
Antiageing agents
Antioxidants
Antiozonants
Plasticizers
Deodorizers
Inhibitors
Passivating agents
Pickling inhibitors
Anticorrosion agents
Antistatics
Stabilizers
Release agents
Lubricants
Flameproofing agents
UV absorbers
Antiknocking agents
Corrosion inhibitors
Metal deactivators
Carburettor cleaning agents
Residue converters
Antiicing agents
Pour point depressors
Defoamers
Lubricity improvers
Optical brighteners

Antifoams:
Anionic surfactants
Polyethylene ethers
Polypropylene glycol ethers
Pluronic®
Mixed ethers

Inorg. peroxides:
Hydrogen peroxide
Lithium peroxide
Sodium peroxide
Calcium peroxide
Strontium peroxide
Barium peroxide

Org. peroxides:
Di-tert-butyl peroxide
Dibenzoyl peroxide
Per-acids
Per-acid esters
Ketone peroxides
Epidioxides
Ascaridol
Ergosterol peroxide

Stabilizers:
Ethylenediaminetetraacetic acid
Magnesium silicate

Plasticizers:
Camphor
Trimellitic acid
Phosphoric acid esters
Azelaic acid esters
Sebacic acid esters
Chloroparaffins
Dioctyl phthalate
Bis-(2-ethylhexyl)phthalate
Diisononyl phthalate
Diisodocecyl phthalate
Phthalic acid esters
Dibutyl phthalate
Diisobutyl phthalate
Dicyclohexyl phthalate
Dimethyl phthalate
Diethyl phthalate
Benzyl butyl phthalate
Butyl octyl phthalate
Butyl deyl phthalate
Dipentyl phthalate
Dimethylglycol phthalate
Dicapryl phthalate
Trimellitic acid esters
Tris-(2-ethylhexyl)trimellitate
Dioctyl adipate
Bis-(2-ethylhexyl)adipate
Diisodecyl adipate
Dibutyl sebacate
Dioctyl sebacate
Bis-(2-ethylhexyl)sebacate
Azelaic acid
Sebacic acid
1,3-Butanediol
1,2-Propanediol
1,4-Butanediol
1,6-Hexanediol
Tricresyl phosphate
Triphenyl phosphate
Diphenyl cresyl phosphate
Diphenyl octyl phosphate
Bis-(2-ethylhexyl)diphenyl phosphate
Tris-(2-ethylhexyl)phosphate
Tris-(2-butoxyethyl)phosphate
Butyl oleate
Butyl stearate
Triethylene glycol bis-(2-ethylbutyrate)
Citric acid esters
Acetyltributyl citrate
Acetyltriethyl citrate
Tartaric acid esters
Lactic acid esters
Epoxystearic acid esters
Epoxidized soya oils
Linseed oils
Benzenesulfonamides
p-Toluenesulfonamides

Free radical interceptors:
Nitrogen monoxide
Bis(trifluoromethyl)nitroxide
Nitroxyl radicals
2,2-Diphenyl-1-picrylhydrazyl
Nitrosobenzene
2-Methyl-2-nitroso-propane
Benzaldehyde tert-butyl nitrone

Wetting agents can be:
Dimethyloctylphosphine oxide
Dimethylnonylphosphine oxide
Dimethyldecylphosphine oxide
Dimethylundecylphosphine oxide
Dimethyldodecylphoshine oxide
N,N,-bis(3-D-gluconamidopropyl)cholamide
N,N-Bis(3-D-gluconamidopropyl)deoxycholamide
Dodecylpoly(oxyethylene glycol ether)s,
PEG (23) dodecyl ether,
PEG (10) cetyl alcohol
PEG (20) cetyl alcohol
PEG (10) stearyl alcohol
PEG (10) oleyl alcohol
PEG (29) oleyl alcohol
Polyethylene glycol (10) lauryl ether
Polyethylene glycol (8) dodecyl ether
Polyethylene glycol (10) isotridecyl ether
Polyethyleneglycol (15) isotridecylether
Ethylphenol-poly(ethylene glycol ether)s
Lubrol
Thesit
Thesit
Cetylpyridinium chloride
Cetyltrimethylammonium bromide
3-[(3-Cholamidopropyl)dimethylammonio]-1-propanesulfonic acid
3-[(3-Cholamidopropyl)dimethylammonio]-1-hydroxypropanesulfonic acid
Chenodeoxycholic acid
Cholate, Na+
Deoxycholate, Na+
Glycocholate, Na+
Glycodeoxycholate, Na+
Taurocholate, Na+
Taurodehydrocholate, Na+
Taurodeoxycholate, Na+
Cyclohexyl-n-ethyl-β-D-maltoside
Cyclohexyl-n-hexyl-β-D-maltoside
Cyclohexyl-n-methyl-β-D-maltoside
n-Decyl-β-D-maltopyranoside
n-Dodecyl-beta-D-maltopyranoside
n-octyl-β-D-maltopyranoside
n-Undecyl-β-D-maltoside
N,N-Dimethyldecylamine oxide
Genaminox KC
N,N-Dimethyldodecylamine oxide
N-Dodecyl-N,N-(dimethylammonio)butyrate
N-Dodecyl-N,N-(dimethylammonio)undecanoate
n-Dodecyl-N,N-dimethylglycine
N-Octyl-N,N-dimethylammonio-3-propanesulfonate
N-Decyl-N,N-dimethylammonio-3-propanesulfonate
N-Dodecyl-N,N-dimethylammonio-3-propanesulfonate
N-Tetradecyl-N,N-dimethylammonio-3-propanesulfonate
Decanoylsucrose
n-Dodecanoylsucrose
Octanoylsucrose
n-Decyl-β-D-glucopyranoside
Dodecyl-β-D-glucopyranoside
n-Heptyl-β-D-glucopyranoside
n-Hexyl-β-D-glucopyranoside
n-Nonyl-β-D-glucopyranoside
n-Octanoyl-β-D-glucosylamine
n-Octyl-beta-D-glucopyranoside
n-Decyl-β-D-thiomaltoside
n-Nonyl-beta-D-thiomaltopyranoside
N,N-Bis(3-D-gluconamidopropyl)deoxycholamide
N,N,-bis(3-D-gluconamidopropyl)cholamide
Digitonin
Bis(2-ethylhexyl)sodium sulfosuccinate
n-Dodecyl-N,N-dimethylglycine
6-O-(N-heptyl-carbamoyl)methyl-α-D-glucopyranoside
N-Dodecanoyl-N-methylglycine
Lauryl-sulfate Li+
Lauryl-sulfate, Na+
{3-([4-tert-Octyl]-1-propanesulfonic acid, Na+
n-Octanoyl-N-methylglucamide
n-Nonanoyl-N-methylglucamide
n-Decanoyl-N-methylglucamide
Ethylphenol-poly(ethylene glycol ether)s
n-Octyl-2-hydroxyethylsulfoxide
n-Octyl-2-hydroxyethyl sulfide
n-Octyl-rac-2,3-dihydroxypropylsulfone
n-octyl-rac-2,3-dihydroxypropylsulfoxide
Polyethylene glycol-polypropylene glycol copolymer
Pluronic F-127
β-D-Fructopyranosyl-alpha-D-glucopyranoside monodecanoate
β-D-Fructopyranosyl-alpha-D-glucopyranoside monododecanoate
PEG (9-10) nonylphenol
PEG (4.5) p-t-octylphenol
PEG (9-10) p-t-octylphenol
PEG (9-10) p-t-octylcyclohexyl
PEG (7-8) p-t-octylphenol
PEG (7-8) t-octylcyclohexyl

Plant protection agents can be:

| Herbicides | Insecticides | Fungicides | |
|---|---|---|---|
| | | | Other |
| 2,4-D | Abamectin | Acibenzolar | Chlormequat |
| 2,4-DB | Acephate | Azoxystrobin | Chloropicrin |
| Acetochlor | Acequinocyl | Benalaxyl | Choline Chloride |
| Acifluorfen | Acetamiprid | Benomyl | Cyclanilide |
| Aclonifen | Acrinathrin | Bitertanol | Dazomet |
| Alachlor | Alanycarb | Bromuconazole | Dichlopropene |
| Alloxidim | Aldicarb | Bupirimate | Dikegulac |
| Ametryn | Alpha- | Captan | Dimethipin |
| | cypermethrin | | |
| Amidosulfuron | Amitraz | Carbendazim | Ethepon |
| Aminotriazole | Azinphos-methyl | Carboxin | Flumetralin |
| Anilofos | Azocyclotin | Carpropamid | Gibberellic acid |
| Asulam | Bacillus | Chlorothalonil | Inabenfide |
| | thuringiensis | | |
| Atrazine | Bendiocarb | Chlozolinate | Maleic hydrazide |
| Azimsulfuron | Benfuracarb | Copper | Mepiquat |
| | | fungicides | |
| Benazolin | Bensultap | Cymoxanil | Metam |
| Benfluralin | Benzoximate | Cyproconazole | Methyl bromide |
| Benfuresate | Bifenazate | Cyprodinil | Methyl |
| | | | isothiocyanate |
| Bensulforon | Bifentrin | Dichlofluanid | Paclobutrazol |
| Bentazone | BPMC (Fenobucarb) | Diclomezine | Prohexadione |
| Benzofenap | Bromopropylate | Diethofencarb | Thidiazuron |
| Bifenox | Buprofezin | Difenoconazole | Triapenthenol |
| Bilanafos | Cadusafos | Dimethirimol | Tributyl |
| | | | phosphorotri- |
| | | | thioate |
| Bispyribac- | Carbaryl | Dimethomorp | Trinexapac-ethyl |
| sodium | | | |
| Bromacil | Carbofuran | Diniconazole | Uniconazole |
| Bromobuthide | Carbosulfan | Dinocap | Fluthiacet - KIH |
| | | | 9201/CGA |
| | | | 248757 |
| Bromofenoxim | Cartap | Dithianon | |
| Bromoxynil | Chinomathionat | Dodemorph | |
| Butachlor | Chlorethoxyfos | Dodine | |
| Butamifos | Chlorfenapyr | Edifenphos | |
| Butralin | Chlorfenvinphos | Epoxiconazole | |
| Butroxydim | Chlorfluazuron | Ethaboxam | |
| Butylate | Chlormephos | Ethirimol | |
| Cafenstrole | Chloropirifos | Etridiazole | |
| Carbentamide | Clofentezine | Famoxadone | |
| Carfentrazone | Cycloprothirin | Fenarimol | |
| Chlorbromuron | Cyfluthrin | Fenbuconazole | |
| Chloridazon | Cyhexatin | Fenhexamid | |
| Chlorimuron | Cypermethrin | Fenitropan | |
| Chlorotoluron | Cyromazine | Fenpiclonil | |
| Chlorsulfuron | Deltamethrin | Fenpropidin | |
| Chlorthal | Demeton-s-methyl | Fenpropimorph | |
| Cinidon-ethyl | Diafenthiuron | Fentin | |
| Cinmethylin | Diazinon | Ferimzone | |
| Cinosulfuron | Dichlorvos | Fluazinam | |
| Clefoxydim | Dicofol | Fludioxonil | |
| Clethodim | Dicrotophos | Fluoroimide | |
| Clodinafop | Diflubenzuron | Fluquinconazole | |
| Clomazone | Dimethoate | Flusilazole | |
| | | | PGR |
| Clomeprop | Disolfoton | Flusulfamide | Aminoethoxy- |
| | | | vinylglycine |
| Clopyralid | Emamectin | Flutolanil | Prohydrojasmon - |
| | benzoate | | PDJ |
| Cloransulam- | Endosulfan | Flutriafol | |
| methyl | | | |
| Cumyluron | Esfenvalerate | Folpet | |
| Cyanazine | Ethiofencarb | Fosetyl | |
| Cyclosulfamuron | Ethion | Fuberidazole | |
| Cycloxidim | Ethoprophos | Furalaxyl | |
| Cyhalofop-butyl | Etofenprox | Furametpyr | |
| Daimuron | Etoxazole | Guazatine | |
| Desmedipham | Etrimfos | Hexaconazole | |
| Desmetryn | Fenamiphos | Hymexazol | |
| Dicamba | Fenazaquin | Imazalil | |
| Dichlobenil | Fenbutatin oxide | Imibenconazole | |
| Dichlorprp | Fenitrothion | Iminoctadine | |
| Diclofop | Fenothiocarb | Ipconazole | |
| Diclosulam | Fenoxycarb | Iprobenfos | |
| Difenzoquat | Fenpropthrin | Iprodione | |
| Diflufenican | Fenpyroximate | Iprovalicarb | |
| Diflufenzopyr | Fenthion | Isoprothiolane | |
| Dimefuron | Fenvalerate | Kasugamycin | |
| Dimepiperate | Fipronil | Kresoxim-methyl | |
| Dimethachlor | Flubroythirinate | Mancozeb | |
| Dimethenamid | Flucycloxuron | Maneb | |
| Diphenamid | Flucythrinate | Mepanipyrim | |
| Diquat | Flufenoxuron | Mepronil | |
| Dithiopyr | Flutenzine | Metalaxyl | |
| Diuron | Fluvalinate | Metconazole | |
| Endothal | Formetanate | Methasulfocarb | |
| EPTC | Formothion | Metiram | |
| Esprocarb | Fosthiazate | Myclobutanil | |
| Ethalfluralin | Furathiocarb | Nitrothal- | |
| | | isopropyl | |
| Ethametsulfuron | Halfenbrox | Nuarimol | |
| Ethofumesate | Halofenozide | Oxadixyl | |
| Ethoxyfen | Hexaflumuron | Oxine-copper | |
| Ethoxysulfuron | Hexythiazox | Oxolinic acid | |
| Etobenzanid | Imidacloprid | Oxycarboxin | |
| Fenoxaprop | Indoxacarb | Pefurazoate | |
| Flamprop-M | Isofenphos | Penconazole | |
| Flazasulfuron | Isoprocarb | Pencycuron | |
| Fluazifop | Isoxathion | Phthalide | |
| Flufenacet | Lambda- | Probenazole | |
| | cyhalothrin | | |
| Flumetsulam | Lindane (Gamma- | Prochloraz | |
| | HCH) | | |
| Flumiclorac- | Lufenuron | Procymidone | |
| pentyl | | | |
| Flumioxazin | Malathion | Propamocarb | |
| Fluometuron | Metaldehyde | Propiconazole | |
| Fluoroglycofen | Methamidophos | Propineb | |
| Flupoxam | Methidathion | Pyrazophos | |
| Flupyrsulfuron | Methiocarb | Pyrifenox | |
| | | | Nematicides |
| Flurenol | Methomyl | Pyrimethanil | ZA3274 |
| Fluridone | Methoprene | Pyroquilon | |
| Flurochloridone | Methoxyfenozide | Quinoxyfen | |
| Fluroxypyr | Mevinphos | Quintozene | |
| Flurtamone | Milbemectin | Spiroxamine | |
| Fomesafen | Monocrotophos | Streptomycin | |
| Glufosinate | Nitenpyram | Sulfur | |
| Glyphosate | Novaluron | Tebuconazole | |
| Halosulforon | Omethoate | Tecloftalam | |
| Haloxyfop | Oxamyl | Tetraconazole | |
| Imazamethabenz | Oxydemeton-methyl | Thiabendazole | |
| Imazamox | Parathion | Thilfluzamide | |
| Imazapic | Parathion-methyl | Thiophanate | |
| | | methyl | |
| Imazapyr | Permethrin | Thiram | |
| Imazaquin | Phenthoate | Tolclofos- | |
| | | methyl | |
| Imazethapyr | Phorate | Tolylfluanid | |
| Imazosulfuron | Phosalone | Triadimefon | |
| Isoprothuron | Phosmet | Triadimenol | |
| Isoxaben | Phosphamidon | Tricyclazole | |
| Isoxaflutole | Phoxim | Tridemorph | |
| Lactofen | Pirimicarb | Triflumizole | |
| Lenacil | Pirimiphos-ethyl | Triforine | |
| Linuron | Pirimiphos-methyl | Triticonazole | |
| MCPA | Profenofos | Validamycin | |
| MCPA-thioethyl | Propaphos | Vinclozolin | |
| MCPB | Propargite | Zineb | |
| Mecoprop | Propoxur | Ziram | |
| Mefenacet | Prothiofos | Cyamidazosulfamid - | |
| | | IKF-916 | |
| Metamitron | Pymetrozine | | |
| Metazachlor | Pyraclofos | Cyamidazosulfamid- | |
| | | IKF-916 | |
| Methabenzthiazuron | Pyridaben | | |
| Methyl-arsonic | Pyridafenthion | Diclocymet - | |
| acid | | S2900 | |
| Metobromuron | Pyrimidifen | Fenamidone - | |
| | | RPA 407213 | |
| Metolachlor | Pyriproxyfen | | |
| Metosulam | Quinakphos | Fenoxanil - | |
| | | AC382042/ | |
| | | NNF9425 | |
| Metoxuron | Silafluofen | | |
| Metribuzin | Spinosad | Iprovalicarb- | |
| | | SZX722 | |
| Metsulforon | Sulprofos | MA 565 | |
| Molinate | Tebufenozide | Metominostrobin - | |
| | | SSF-126 | |
| Naproanilide | Tebufenpyrad | | |
| Napropamide | Tebupirimfos | MTF-753 | |
| Naptalam | Teflubenzuron | NF-149 | |
| Nicosulfuron | Tefluthrin | NNF-9850 | |
| Norflurazon | Terbufos | Oxpoconazole | |
| | | fumarate - UBF- | |
| | | 910 | |
| Orbencarb | Thiamethoxam | | |
| Oryzalin | Thiocyclam | Picoxystrobin - | |
| | | ZA1963 | |
| Oxadiargyl | Thiodicarb | | |
| Oxadiazon | Thiometon | | |
| Oxasulfuron | Tralomethrin | Silthiopharm - | |
| | | MON-65500 | |
| Oxyfluorfen | Triazamate | | |
| Paraquat | Triazophos | Simeconazole - | |
| | | F155 | |
| Pendimethalin | Trichlorfon | Trifloxystrobin - | |
| | | OGA279202 | |
| Pentoxazone | Triflumuron | | |
| Phenmedipham | Vamidothion | Zoxamide - | |
| | | RH7281 | |
| Picloram | Xylyl | | |
| | methylcarbamate | | |
| Pretilachlor | Zeta-Cypermethrin | | |
| Primisulfuron | Acetoprole- | | |
| | RPA115782 | | |
| Prometryn | AKD 1022 | | |
| Propachlor | Chromafenozide- | | |
| | ANS-118 | | |
| Propanil | | | |
| Propaquizafop | Clothianidin - | | |
| | TI-435 | | |
| Propazine | Dinitefuran - | | |
| | MTI-446 | | |
| Propyzamide | Ethiprole-RPA | | |
| | 107382 | | |
| Prosulfocarb | Fluacrypyrim - | | |
| | NA-83 | | |
| Prosulfuron | Flupyrazofos | | |
| Pyraflufen- | Phosphocarb - | | |
| ethyl | BAS301 | | |
| Pyrazolinate | | | |
| Pyrazosulfuron | Protrifenbute - | | |
| | FMC 111869 | | |
| Pyrazoxyfen | | | |
| Pyribenzoxim | Thiacloprid - | | |
| | BAYYRC2894 | | |
| Pyributicarb | | | |
| Pyridate | Tolfenpyrad - | | |
| | OMI-88 | | |
| Pyriminobac- | | | |
| methyl | | | |
| Pyrithiobac | | | |
| Quinclorac | | | |
| Quinmerac | | | |
| Quinoclamine | | | |
| Quizalofop | | | |
| Quizalofop-P- | | | |
| tefuryl | | | |
| Rimsulfuron | | | |
| Sethoxydim | | | |
| Simazine | | | |
| Sulcotrione | | | |
| Sulfentrazone | | | |
| Sulfometuron | | | |
| Sulfosate | | | |
| Sulfosulfuron | | | |
| Tebuthiuron | | | |
| Terbacil | | | |
| Terbumeton | | | |
| Terbuthylazine | | | |
| Terbutryn | | | |
| Thenylchlor | | | |
| Thiazopyr | | | |
| Thifensulfuron | | | |
| Thiobencarb | | | |
| Herbicides | | | |
| Tralkoxydim | | | |
| Triallate | | | |
| Triasulfuron | | | |
| Tribenuron | | | |
| Triclopyr | | | |
| Trifluralin | | | |
| Triflusulfuron | | | |
| Amicarbazone-BAYMKH3586 | | | |
| Azafenidin-DPX-R6447 | | | |
| Beflubutamid-UBH-820 | | | |
| Benzfendizone - FMC 143686 | | | |
| Benzobicyclon - SB-500 | | | |
| Butafenacil - CGA 276854 | | | |
| Fentrazamide - BAYYRC2388 | | | |
| Florasulam - DE570 | | | |
| Fluazolate - JV485 | | | |
| Flucarbazone - BAYMKH6562 | | | |
| Flufebpyr-ethyl - S-3453 | | | |
| Foramsulfuron - AEF 130360 | | | |
| Indanofan - MK-243 | | | |
| Iodosulfuron - AEF 115008 | | | |
| Isoxadifen - AEF122006 | | | |
| KPP421 | | | |
| Mesosulfuron - AEF 130060 | | | |
| Mesotrione - ZA1296 | | | |
| MTB-951 | | | |
| OK-9701 | | | |
| Oxaziclomefone-MY-00 | | | |
| Penoxsulam - DE638 | | | |
| Pethoxamid - TKC-94 | | | |
| Picolinofen - AC900001 | | | |
| Propoxycarbazone (proposed) | | | |
| BAYMKH6561 | | | |
| Pyriftalid - CGA279233 | | | |
| Tepraloxydim - BAS620H/NP61EC | | | |
| Triaziflam - IDH 1105 | | | |
| Trifloxysulfuron (Na salt) - | | | |
| CGA362622 | | | |
| Tritosulfuron | | | |

Preferably, however, the silicon dioxide granules employed according to the invention function as a carrier. The present invention therefore also relates to an adsorbate of the silicon dioxide granules described above and at least one of these substances.

The term “adsorbate” as used herein includes the adsorption of a substance not only on to the surface of the silicon dioxide, but also into the pores, as well as the “intercalation” into the intergrain volumes. “Adsorbate” can also mean that silicon dioxide granules or fragments thereof envelop solid particles or liquid droplets of the substance. In the latter case the forces of attraction between the particles or droplets are reduced and, for example, the flow properties are improved or the merging of droplets is impeded.

The ratio of amounts of substance to silicon dioxide granules in the adsorbate can be chosen as desired as a function of the properties of the substance and the requirements for the end product. Preferably, however, 0.001 to 200 g of substance are employed per 100 g of silicon dioxide granules, particularly preferably 10 to 150 g.

In a preferred embodiment, granules based on pyrogenically prepared silicon dioxide of average particle diameter from 10 to 120 μm and BET surface area from 40 to 400 m²/g (determination in accordance with DIN 66 131 with nitrogen) can be used as the silicon dioxide granules.

The silicon dioxide granules furthermore preferably have the following physico-chemical characteristic data, which are determined as described in EP PS 0 725 037:

Pore volume: 0.5 to 2.5 ml/g

Pore size volume: less than 5% of the total pore volume has a pore diameter of less than 5 nm, remainder meso- and macropores

pH: 3.6 to 8.5

Tamped density: 220 to 700 g/l.

Granules which are suitable for the use according to the invention and the preparation thereof are described, for example, in EP OS 0 727 037.

An example of a process for the preparation of the adsorbate according to the invention comprises:

Melting of the substance(s) to be adsorbed, chosen from foodstuffs additives, such as dyestuffs, antioxidants, preservatives, emulsifiers, gelling agents, thickeners and binders, stabilizers, alkalis, acids, salts, antilumping agents, flavour intensifiers, sweeteners, aromas, feedstuffs additives, chemical intermediates and plant protection agents, such as, for example, herbicides, insecticides and fungicides, or distribution, i.e. dissolving, suspending or emulsifying, thereof in a solvent;

mixing of the granules based on pyrogenically prepared silicon dioxide with the mixture from step (a); and where appropriate removal of the solvent.

“Solvent” also includes mixtures of several different solvents. It goes without saying, furthermore, that substances which are already liquid at room temperature can be subjected to the mixing in step (b) without prior processing, since in this case the “melting operation” has already taken place. Mixing step (b) can be carried out either by adding the mixture from step (a) to the silicon dioxide granules, for example by spraying on, or vice versa. In both cases, the addition can be made in one amount or in portions. The duration of the mixing in step (b) depends here above all on the adsorption properties of the substance to be adsorbed on the silica surface. If a solvent is present, step (a) and (b) are carried out at a temperature which lies between the freezing and boiling point of the solvent. The solvent, where appropriate in excess, is preferably removed in step (c) at elevated temperature and/or under reduced pressure.

The removal of the solvent in step (c) can also be carried out by spray drying or fluidized bed drying, shaping taking place at the same time. In the case of a granule-containing melt, the shaping process can accordingly be an extrusion.

The adsorbates according to the invention can be used for the preparation of powders, liquids, foams, sprays, gels, creams, ointments, pastes, sticks and tablets.

The adsorbates according to the invention can additionally be shaped. They can be processed, for example, to pellets, larger granules, extrudates etc.

The advantage of the adsorbates according to the invention lies in their excellent flow properties, the low water content and the high purity of the starting granules. They offer a very good possibility for dispersing substances which are difficult to meter, and are easy to handle.

When handling the adsorbates, the hazard potential to the administering person during use on toxic substances, such as plant protection agents or aggressive skin-irritating substances, can be reduced significantly.

When the adsorbate according to the invention is used, a uniform distribution of the active compound can be achieved.

The invention is now to be explained in more detail with the aid of examples.

Preparation of Granules Based on Pyrogenically Prepared Silicon Dioxide

The pyrogenically prepared silicon dioxide AEROSIL 300, commercially obtainable from Degussa AG, is used as the starting compound.

The pyrogenically prepared silicon dioxide is dispersed in completely demineralized water. A dispersing unit which operates by the rotor/stator principle is used here. The suspension formed is spray dried. The finished product is separated off via a filter or cyclone. The heat treatment of the spray granules is carried out in a muffle oven.

The preparation parameters are shown in table 1.

TABLE 1

| Starting SiO₂ | AEROSIL 300 |
|---|---|
| Spray drying data | |
| Amount of H₂O (kg) | 100 |
| Amount of SiO₂ (kg) | 10 |
| Atomization with | disc atomizer |
| Operating temperature (° C.) | 480° C. |
| Waste air temperature (° C.) | 103° C. |
| Separation | filter |
| Physico-chemical data | |
| BET surface area (m²/g) | 298 |
| Particle size d⁵⁰ (μm) | 30 |
| Tamped volume (g/l) | 283 |
| pH | 4.7 |

### EXAMPLES

1. Starting Materials

1.1 Model Liquids

Vitamin E acetate, silicone oil, paraffin oil and eucalyptus oil are used as model liquids for the fields of use according to the invention. Vitamin E acetate is used, for example, in the nutrition of animals and humans, and eucalyptus oil as an aromatic or aroma substance.

| Example | Product | Product name | Manufacturer |
|---|---|---|---|
| 1 | Vitamin E | | BASF |
| | acetate | | |
| 2 | Silicone oil | Silicon Fluid 345 | Dow Corning |
| 3 | Paraffin oil | Paraffinöl | Merck |
| | | dünnflüssig | |
| 4 | Eucalyptus | Oleum Eucalypti | Caelo |
| | oil | 80-85% | |

1.2 Carrier Silicas

| | Loss on | Loss on | SiO₂ | | Bulk |
|---|---|---|---|---|---|
| | drying | ignition | content | Slope | density |
| Silica | (wt. %) | (wt. %) | (wt. %) | angle (°) | (g/l) |
| Example | 1.7 | 2.1 | 99.9 | 34.97 | 232.8 |
| 1-4 | | | | | |
| AEROPERL ® | | | | | |
| 300/30 | | | | | |
| (Degussa) | | | | | |
| Comparison | 4.8 | 4.4 | 98.0 | 38.99 | 211 |
| example 1 | | | | | |
| SIPERNAT ® | | | | | |
| 22 | | | | | |
| (Degussa) | | | | | |
| Comparison | 4.5 | 4.9 | 98.5 | 52.67 | 136.67 |
| example 2 | | | | | |
| SIPERNAT ® | | | | | |
| 50 | | | | | |
| (Degussa) | | | | | |
| Comparison | 5.9 | 3.9 | nd | 50 | 92 |
| example 3 | | | | | |
| Syloid 244 | | | | | |
| FP | | | | | |
| (Grace) | | | | | |

Granulated pyrogenic silica (AEROPERL® 300/30) has a significantly lower water content (loss on drying and ignition) and a higher silicon dioxide content than the silicas used in the comparison examples. Furthermore, it is free from sulfates, typical impurities of precipitated silica and silica gels, and has the best flowability (the lowest slope angle).

2. Procedure:

50 g of carrier silica are initially introduced into a 2 litre three-necked flask equipped with a blade stirrer 50 g of the model liquids from examples 1-4 are added dropwise from a dropping funnel in the course of 60 minutes, while stirring at a stirrer speed of 100 revolutions/minute. Comparison examples 1-3 are carried out with eucalyptus oil. The liquid-silica adsorbates are then sieved manually three times through a 0.8 mm sieve and left to stand overnight in a closed screw-cap glass bottle. The following day, the liquid-silica adsorbates are characterized by the following methods:

Flow rating by means of glass flow vessels in accordance with the publication series Pigmente No. 31 “AEROSIL zur Verbesserung des Flieβverhaltens pulverförmiger Substanzen”, Degussa AG, Dusseldorf.

Poured cone height (cm) or slope angle (°) in accordance with the publication series Pigmente No. 31. The slope angle is obtained from the poured cone height via the equation
tan(slope angle)=(poured cone height/0.5 cone diameter)
Bulk density (g/l) in accordance with DIN standard 6613.

3. Results

| | Flow | Slope angle | Bulk density |
|---|---|---|---|
| | rating | (°) | (g/l) |
| Example 1 | 2 | 30.1 | 501 |
| AEROPERL/Vitam. E | | | |
| Example 2 | 2 | 37.2 | 475 |
| AEROPERL/Silicone | | | |
| oil | | | |
| Example 3 | 2 | 38.7 | 497 |
| AEROPERL/Paraffin | | | |
| oil | | | |
| Example 4 | 2 | 37.2 | 594 |
| AEROPERL/ | | | |
| Eucalyptus oil | | | |
| Comparison example 1 | 3 | 46.0 | 450 |
| SIPERNAT 22 | | | |
| Comparison example 2 | 4 | 63.9 | 353 |
| SIPERNAT 50 | | | |
| Comparison example 3 | 6 | 56.7 | 201 |
| Syloid FP 244 | | | |

The liquid-silica adsorbates prepared with granulated pyrogenic silica (AEROPERL® 300/30) are distinguished by a good flowability (flow rating 2, slope angle<40° C.). In contrast, the liquid-silica adsorbates from comparison examples 1 to 3 show a significantly lower flowability. The latter moreover have significantly lower bulk densities.

Liquid-silica adsorbates with a good flowability and high bulk volume are advantageous for carrier uses. Furthermore, carrier silicas should have the lowest possible water content and should be very pure, in order to avoid decomposition of the adsorbed liquids under the (catalytic) influence of water or impurities, such as, for example, sulfates. The experiments show that granulated pyrogenic silica meets all these requirements.

1. Use of granules based on pyrogenically prepared silicon dioxide as a carrier for substances chosen from the group consisting of foodstuffs additives, such as dyestuffs, antioxidants, preservatives, emulsifiers, gelling agents, thickeners and binders, stabilizers, alkalis, acids, salts, antilumping agents, flavour intensifiers, sweeteners, aromas, feedstuffs additives, chemical intermediates and plant protection agents, such as, for example, herbicides, insecticides and fungicides.

2. Use according to claim 1, characterized in that the silicon dioxide granules are silanized.

3. Adsorbate of granules based on pyrogenically prepared silicon dioxide and at least one substance chosen from the group consisting of foodstuffs additives, such as dyestuffs, antioxidants, preservatives, emulsifiers, gelling agents, thickeners and binders, stabilizers, alkalis, acids, salts, antilumping agents, flavour intensifiers, sweeteners, aromas, feedstuffs additives, chemical intermediates and plant protection agents, such as herbicides, insecticides and fungicides.

4. Adsorbate according to claim 3, characterized in that the silicon dioxide granules are silanized.

5. Dyestuff comprising granules based on pyrogenically prepared silicon dioxide.

6. Antioxidant comprising granules based on pyrogenically prepared silicon dioxide.

7. Preservative comprising granules based on pyrogenically prepared silicon dioxide.

8. Emulsifier comprising granules based on pyrogenically prepared silicon dioxide.

9. Gelling agent comprising granules based on pyrogenically prepared silicon dioxide.

10. Thickener comprising granules based on pyrogenically prepared silicon dioxide.

11. Binder comprising granules based on pyrogenically prepared silicon dioxide.

12. Stabilizer comprising granules based on pyrogenically prepared silicon dioxide.

13. Alkali comprising granules based on pyrogenically prepared silicon dioxide.

14. Acids comprising granules based on pyrogenically prepared silicon dioxide.

15. Salts comprising granules based on pyrogenically prepared silicon dioxide.

16. Antilumping agent comprising granules based on pyrogenically prepared silicon dioxide.

17. Flavour intensifier comprising granules based on pyrogenically prepared silicon dioxide.

18. Sweetener comprising granules based on pyrogenically prepared silicon dioxide.

19. Aroma comprising granules based on pyrogenically prepared silicon dioxide.

20. Feedstuffs additives comprising granules based on pyrogenically prepared silicon dioxide.

21. Chemical intermediates comprising granules based on pyrogenically prepared silicon dioxide.

22. Plant protection agents comprising granules based on pyrogenically prepared silicon dioxide.

23. Herbicides comprising granules based on pyrogenically prepared silicon dioxide.

24. Insecticides comprising granules based on pyrogenically prepared silicon dioxide.

25. Fungicides comprising granules based on pyrogenically prepared silicon dioxide.
